(19) United States
(12) Patent Application Publication
(10) Pub. No.: US 2022/0304953 A1
(43) Pub. Date: Sep. 29, 2022

(54) PHARMACEUTICAL DOSAGE FORM COMPRISING METFORMIN AND CALCIUM CITRATE

(71) Applicant: DSM IP ASSETS B.V., Heerlen (NL)

(72) Inventors: Martin Thomas KUENTZ, Kaiseraugst (CH); Zdravka MISIC, Kaiseraugst (CH); Ralph SCHNEITER, Kaiseraugst (CH); Araksya TOPCHYAN, Kaiseraugst (CH)

(21) Appl. No.: 17/619,485

(22) PCT Filed: Jun. 17, 2020
(86) PCT No.: PCT/EP2020/066762

§ 371 Date: Dec. 15, 2021

(30) Foreign Application Priority Data
Jun. 17, 2019 (EP) 19180452.5

(51) Int. Cl. A61K 31/155; A61K 31/714; A61K 47/12; A61K 9/20; A61P 25/02

(52) U.S. Cl. CPC A61K 31/155; A61K 31/714; A61K 47/12; A61K 9/2013; A61P 25/02

(57) ABSTRACT

The present invention relates to a pharmaceutical dosage form comprising a) metformin or a pharmaceutical acceptable salt thereof, and b) calcium citrate. In a preferred embodiment of the invention, the dosage form further comprises vitamin B12. The invention also relates to its use in the prevention or alleviation of metformin induced vitamin B12 malabsorption.

### TECHNICAL FIELD

The present invention relates to the use of metformin for the treatment of type 2 diabetes.

### BACKGROUND OF THE INVENTION

During long term use of metformin, vitamin B12 absorption might decrease. The Product Monograph of GLUCOPHAGE®, for example, contains the following warning:
“Long-term treatment with GLUCOPHAGE® has been associated with a decrease in serum vitamin B12 levels which may cause peripheral neuropathy. Serious cases of peripheral neuropathy have been reported with GLUCOPHAGE® treatment in the context of vitamin B12 deficiency (see ADVERSE REACTIONS, Post-Market Adverse Drug Reactions). Monitoring of serum vitamin B12 levels is recommended.”

GLUCOPHAGE® is just one brand under which metformin hydrochloride tablets are being marketed. Similar warnings can be found in package leaflets of other drug manufacturers.

Bauman et al. have shown that an increased intake of calcium carbonate can reverse metformin induced vitamin B12 malabsorption (Diabetes Care, Volume 23, Number 9, September 2000, pages 1227-1231). In the Bauman study, patients were given two kinds of tablets: the usual metformin tablet and, in addition, a dietary supplement comprising calcium carbonate.

Whereas dietary supplements are easily accessible, it is known that patient compliance is notoriously low when two tablets instead of one only needs to be taken on a regular basis. This is particularly true for elderly diabetes patients as there is often further co-medication in diabetes patients. Co-medication increases the so-called “pill count” (i.e. number of tablets to be administered) and hence the risk of poor adherence to therapy.

Patient compliance becomes even worse if one tablet comprises a prescription drug (Rx), whereas the second tablet may be bought in the supermarket. So far, patients are just not used to the combined intake of tablets that originate from very different supply channels.

In addition, symptoms from vitamin B12 deficiency occur rather slowly and may be indistinguishable from symptoms caused by chronically high blood sugar and diabetes. Therefore, the omittance of one or two calcium carbonate supplements will not instantly worsen the patient's condition. This also negatively affects patient's compliance as the perception of not feeling much relief during the course of taking the medication influences the patient's decision to discontinue the treatment regimen.

The problem to be solved by the present invention is to improve patient's compliance when applying the treatment regimen suggested by Bauman et al. (Diabetes Care 23:1227-1231, 2000).

Thus, there is a need for a fixed-dose combination (FDC) which prevents, or reverses metformin induced vitamin B12 malabsorption, and which improves patient compliance, has no or little side effects, is storage stable, is easy to swallow, is easy to manufacture, and meets quality standards of the pharmaceutical industry.

### SUMMARY OF THE INVENTION

Ionic calcium is obligatory for the B12-IF complex to attach to ileal cell surface receptors. Because metformin competes with calcium for the mucosal cell membrane, vitamin B12 malabsorption is at least partially reversible with ionic calcium.

Calcium ions must have a solid form to be compressed into a tablet. Therefore, a calcium salt comprising calcium ions is used. Many calcium salts are known. In foods, calcium salts such as calcium lactate, calcium diphosphate, and tricalcium phosphate are used. Calcium lactobionate is a white powder that is used as a suspending agent for pharmaceuticals. Calcium stearate is a known lubricant, and in baking, calcium monophosphate is used as a leavening agent. The list of calcium salts for potential oral use also includes compounds such as calcium sulfite, calcium silicate, and calcium acetate.

The problems underlying the present invention are solved by choosing a calcium salt of citric acid as a source for the required calcium ions.

Different kinds of calcium citrates are known. Examples are anhydrous calcium citrate or a hydrated form thereof such as tricalcium dicitrate tetrahydrate or calcium citrate hexahydrate.

In the context of the present invention, tricalcium dicitrate tetrahydrate is preferred. Thus, a preferred embodiment of the present invention relates to an oral fixed-dose combination (FDC) comprising tricalcium dicitrate tetrahydrate and metformin or a pharmaceutical acceptable salt thereof. A particularly preferred embodiment of the present invention relates to an oral fixed-dose combination (FDC) comprising tricalcium dicitrate tetrahydrate and metformin hydrochloride.

Various kinds of fixed-dose combinations are known. In the context of the present invention, the fixed-dose combination is preferably a solid oral dosage form such as a capsule or a tablet.

For the manufacture of a solid oral dosage form, the selection of calcium citrate is surprising because it results, at least at first glance, in a very large solid oral dosage form. Such large tablets or capsules do not improve patient compliance as they are difficult to swallow. The described unwanted increase in size is particularly pronounced if tricalcium dicitrate tetrahydrate is used as calcium citrate.

Said unwanted increase in volume/size is due to the relatively small amount of calcium ions per gram calcium citrate, compared to calcium carbonate. Calcium carbonate is the calcium salt that has been administered in the Bauman study.

One gram of calcium carbonate (CaCO₃; 100.09 g/mol) comprises a significantly larger number of calcium ions (in mol) than one gram of tricalcium dicitrate tetrahydrate ([Ca₃(C₆H₅O₇)₂(H₂O)₂].2H₂O, 570.5 g/mol). This is of a relevance because a relatively large amount of oral calcium carbonate (1.2 g/day, corresponding to 0.012 mol Ca²⁺) was administered in the Bauman study. To administer 0.012 mol Ca²⁺, 6.8 g tricalcium dicitrate tetrahydrate would have to be compressed into a tablet or would have to be filled into a capsule, together with the usual amount of metformin.

This seems hardly possible.

The inventors have found a surprising way to provide a reasonably small solid oral dosage form that comprises calcium citrate and which prevents or reverses metformin induced vitamin B12 malabsorption.

A preferred embodiment of the present invention relates to a tablet or to a capsule comprising tricalcium dicitrate tetrahydrate and metformin hydrochloride.

Dual Functionality of Calcium Citrate

Surprisingly, the addition of calcium citrate leads to tablet hardening without negatively effecting the disintegration time of the tablet. Thus, in the context of the present invention, calcium citrate has a dual functionality: a medical function (e.g. at least partial reversal of vitamin B12 malabsorption) and a galenical function (as tablet hardener). In other words, adding calcium citrate to metformin results in a tablet that reverses or reduces vitamin B12 malabsorption and has at the same time optimal tablet hardness.

Due to the dual functionality of calcium citrate, other excipients can be omitted or their amount in the tablet can be reduced. Thus, the disadvantage of calcium citrate (i.e. the large tablet size due to the limited amount of Ca²⁺ per gram calcium citrate) can be partially compensated by omitting or reducing other excipients that are commonly added to facilitate tablet compaction. Each excipient that does not need to be added makes the tablet smaller, simplifies the manufacturing process and/or lowers the cost of goods sold (COGS).

Thus, the present invention also relates to the use of calcium citrate as a dual functional compound, wherein the two functions are (i) increasing tablet hardness and (ii) preventing or reversing metformin induced vitamin B12 malabsorption.

High Solubility and Bioavailability of Calcium Citrate

Bauman et al. teaches to administer 1.2 g calcium carbonate per day. In theory, this corresponds to an intake of 0.012 mol Ca²⁺. However, in practice, less than 0.012 mol Ca²⁺ will be available to the patient as calcium carbonate has a limited solubility.

Calcium citrate has a higher solubility than calcium carbonate. In addition, it has been shown that calcium citrate has superior bioavailability than calcium carbonate (Tondapu et al., Comparison of the Absorption of Calcium Carbonate and Calcium Citrate after Roux-en-Y Gastric Bypass, OBES SURG (2009) 19:1256).

The high solubility and/or bioavailability of calcium citrate allows to reduce the amount of calcium salt in the oral dosage form. Thus, it is sufficient to compress less than 6.8 g tricalcium dicitrate tetrahydrate into a tablet to obtain the same or at least a similar effect as adding 1.2 g calcium carbonate (corresponding to the 0.012 mol Ca²⁺ in the Bauman study). And as a result of the reduced amount of tricalcium dicitrate tetrahydrate, the tablet size becomes acceptable for convenient oral administration.

Thus, a preferred embodiment of the present invention relates to a solid oral dosage form comprising calcium citrate and metformin or a pharmaceutical acceptable salt thereof, wherein said solid oral dosage form comprises less than 0.012 mol Ca²⁺, preferably less than 0.006 mol Ca²⁺ and most preferably less than 0.001 mol Ca²⁺.

Leverage Up the Medical Function of Calcium Citrate

Liver, fish, cheese and other food contain vitamin B12. However, even if the patient's nutrition includes food which contains vitamin B12, after long term use of metformin, the patient's vitamin B12 serum level may eventually decrease. This is due to the malabsorption of vitamin B12. Malabsorption means that significantly less than 100% of the food's vitamin B12 is absorbed.

One manner to increase the patient's vitamin B12 serum level is to improve the absorption of vitamin B12 from the food. As shown by Bauman et al., this can be done by the oral intake of calcium carbonate.

In a preferred embodiment of the invention, the medical effect of Ca²⁺ is leveraged up by the concomitant administration of vitamin B12. Thus, instead of only improving the absorption of vitamin B12, the daily intake of vitamin B12 is also increased. In this embodiment of the invention, it is acceptable to further reduce the amount of calcium citrate because if the malabsorption of vitamin B12 was only partially reversed, the gap to full reversal of malabsorption would be filled by the increased intake of vitamin B12. The benefit of a reduced amount of calcium citrate is the smaller size of the solid oral dosage form; and because smaller tablets/capsules are easier to swallow, patient compliance is improved.

Thus, the present invention also relates to the method for reducing the size of a tablet which comprises metformin and calcium citrate, wherein the tablet's calcium citrate is partially replaced by a vitamin B12. It also relates to the method for reducing the size of a capsule which comprises metformin and calcium citrate, wherein the capsule's calcium citrate is partially replaced by a vitamin B12. The preferred pharmaceutical dosage form of the present invention is a tablet or a capsule and comprises from 0.0001 to 0.012 mol Ca²⁺, preferably from 0.0003 to 0.006 mol Ca²⁺ and most from 0.0005 to 0.001 mol Ca²⁺.

Toward Achieving Optimal Response: Managing Potential Side Effects

Nowadays, patients inform themselves via the internet, and a search for “calcium carbonate” will reveal that a regular intake of calcium carbonate might possibly increase the risk for kidney stones. Regardless whether an internet search reveals the truth, it would have an impact on patient's compliance.

Calcium citrate is not only a surprising multifunctional compound, it is also expected to be well perceived by patients because calcium citrate has no history of triggering kidney stones. In contrast: urinary citrate is known to reduce urinary supersaturation of calcium oxalate and phosphate, calcium oxalate and calcium phosphate being amongst the most common crystalline materials found in kidney stones (Usui et al., Urinary Citrate in Kidney Stone Disease Tokai J Exp Clin Med., Vol. 28, No. 2, pp. 65-70, 2003).

Thus, the present invention also relates to the use of calcium citrate to eliminate or to reduce the risk of developing kidney stones when being treated with a pharmaceutical dosage form which comprises metformin or a pharmaceutical salt thereof and at least one source of calcium ions.

### FIGURES

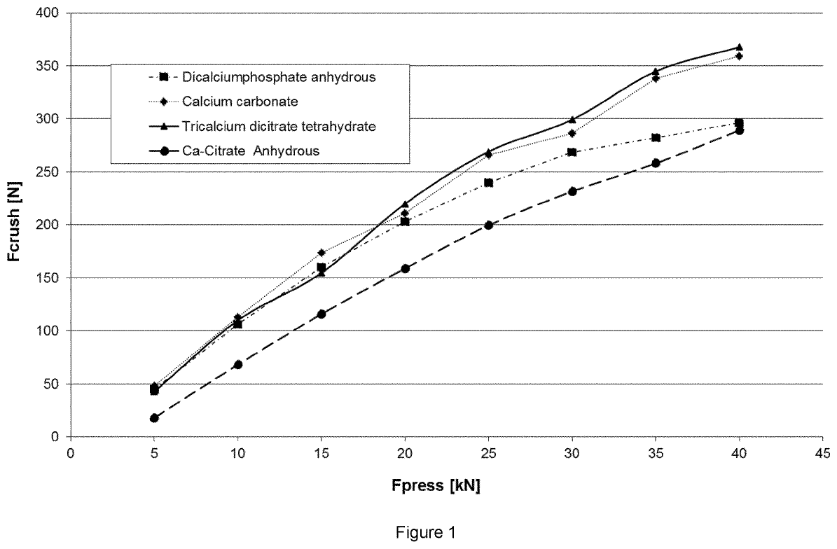

In order to test the influence of the different sources of ionic calcium, four similar tablets were prepared. FIG. 1 shows the compression profiles of the four tablets. Fcrush is the force needed to break a tablet axially. Fpress is the force developed by upper punch during tableting.

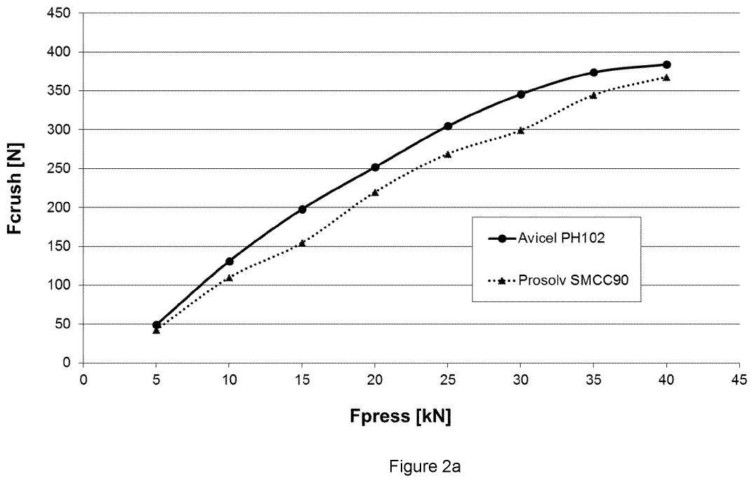

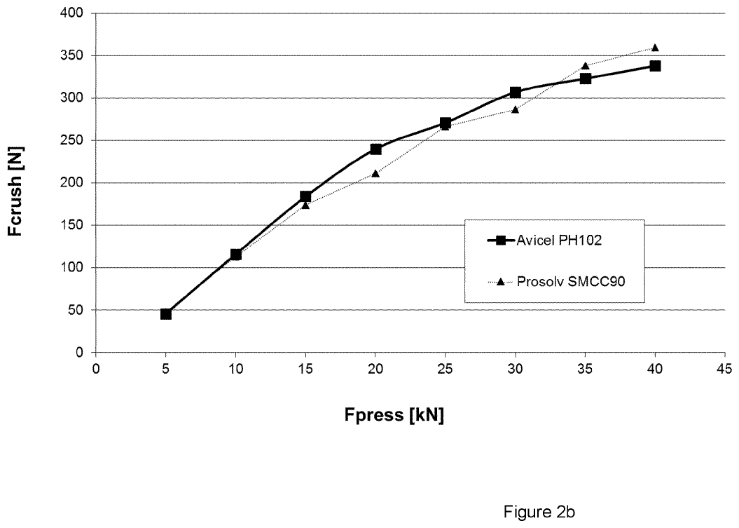

In order to test the influence of two different binders, two similar tablets were prepared. As a source of ionic calcium, both tablets comprised tricalcium dicitrate tetrahydrate. FIG. 2a shows the compression profiles of the two tablets.

Again, in order to test the influence of two different binders, two similar tablets were prepared. This time, however, the two tablets comprised calcium carbonate as a source of ionic calcium. FIG. 2b shows the compression profiles of the two tablets.

### DETAILED DESCRIPTION OF THE INVENTION

A preferred embodiment of the invention relates to a pharmaceutical dosage form comprising calcium citrate, metformin and vitamin B12. Calcium citrate has excellent solubility in the stomach and shows less or almost no precipitation in the ileum. The simultaneous oral administration of calcium citrate and vitamin B12 prevents or reverses metformin induced vitamin B12 deficiency in a particularly effective manner. Particularly good patient compliance is achieved by providing a fixed-dose combination comprising all three ingredients, i.e. calcium citrate, metformin and vitamin B12.

### Definitions

In the context of the present invention, the term “calcium citrate” refers to any calcium salt of citric acid. Thus, the term includes monocalcium citrate, dicalcium citrate and tricalcium citrate. In a preferred embodiment of the invention, the term “calcium citrate” refers to any kind of tricalcium citrate salt.

Known tricalcium citrate salts include anhydrous calcium citrate (i.e. Ca₃(C₆H₅O₇)₂) and tricalcium dicitrate tetrahydrate (i.e. [Ca₃(C₆H₅O₇)₂(H₂O)₂].2H₂O). Therefore, the term “calcium citrate” refers preferably to anhydrous calcium citrate, tricalcium dicitrate tetrahydrate or a mixture thereof. In the context of the present invention, “Ca²⁺” is referred to as ionic calcium or as calcium ion(s).

Vitamin B12 is a well-known water-soluble vitamin. In the context of the present invention, the term “vitamin B12” refers to any vitamer of vitamin B12 and includes vitamin B12 derivatives and/or metabolites of vitamin B12. Preferably, however, the term “vitamin B12” refers to cyanocobalamin. Cyanocobalamin may be produced by fermentation using suitable microorganisms.

In the context of the present invention, crystalline vitamin B12 may be used. Crystalline vitamin B12 is commercially available.

In a preferred embodiment of the present invention, however, a spray dried formulation of vitamin B12 is used. The expression “spray dried formulation of vitamin B12” refers to a powder which is obtainable by spray drying of an aqueous solution that comprises vitamin B12 and at least one excipient, wherein said at least one excipient is preferably selected from the group consisting of sodium citrate, trisodium citrate, citric acid, maltodextrin citric acid and modified food starch. In a preferred embodiment of the invention, the expression “spray dried formulation of vitamin B12” refers to a powder which is obtainable by spray drying an aqueous solution which comprises cyanocobalamin and at least one excipient, wherein said at least one excipient is preferably selected from the group consisting of sodium citrate, trisodium citrate, citric acid, maltodextrin and modified food starch. Typically, the expression “spray dried formulation of vitamin B12” refers to a water-soluble or water-dispersible powder which comprises from 0.01 to 1 weight-%, preferably from 0.05 to 0.5 weight-% and most preferably 0.1 weight-% cyanocobalamin, based on the total weight of the spray dried formulation of vitamin B12. Thus, in the most preferred embodiment of the invention, the expression “spray dried formulation of vitamin B12” refers to a powder which is obtainable by spray drying an aqueous solution which comprises cyanocobalamin and at least one excipient, wherein said excipient is preferably selected from the group consisting of sodium citrate, trisodium citrate, citric acid, maltodextrin and modified food starch, and wherein said powder comprises 1 weight-% or less of cyanocobalamin, based on the total weight of the powder.

Metformin is a well-known pharmaceutical drug. In the context of the present invention, the term “metformin” may refer to metformin or to a pharmaceutical acceptable salt thereof. The probably best known pharmaceutical acceptable salt of metformin is metformin HCl. Therefore, in the most preferred embodiment of the invention, the term “metformin” refers to metformin HCl. The pharmaceutical dosage form of the invention comprises preferably 500 mg metformin HCl or 1000 mg metformin HCl. In a preferred embodiment of the invention, the pharmaceutical dosage form is a tablet or a capsule which comprises 1000 mg metformin HCl.

Metformin has a poor compactibility and flowability. Therefore, metformin is preferably granulated before tableting. During such granulation process, metformin is transformed into free-flowing, essentially dust-free granules that are easy to compress. In the context of the present invention, the term “granulated metformin” refers to granules comprising metformin or pharmaceutical salt thereof. Preferably, the term “granulated metformin” refers to granules comprising at least 50 weight-% metformin, based on the total weight of the granules, and at least one excipient, wherein said excipient is preferably a binder and/or a lubricant. Suitable binders are listed for example in Arndt et al., “Roll Compaction and Tableting of High Loaded Metformin Formulations Using Efficient Binders”, AAPS PharmSciTech, July 2018, Volume 19, Issue 5, pp 2068-2076. In the context of the present invention, the term “granulated metformin” includes granulated pharmaceutical acceptable salts of metformin such as granulated metformin HCl. Therefore, the term “granulated metformin” may refer to granules comprising at least 50 weight-% metformin HCl, based on the total weight of the granules, and at least one excipient, wherein said excipient is preferably a binder and/or a lubricant. Granulated metformin is commercially available. Metformin granulate DC grade 92.6% as available at Vistin Pharma (Oslo, Norway) comprises magnesium stearate as lubricant. Therefore, in the most preferred embodiment of the invention, the term “granulated metformin” refers to granules comprising magnesium stearate and at least 90 weight-% metformin HCl, based on the total weight of the granules.

In the context of the present invention, the term “pharmaceutical dosage form” refers preferably to an oral dosage form. Whereas liquid oral dosage forms of metformin are known (e.g. Riomet® in India), the term “pharmaceutical dosage form” refers preferably to a solid oral dosage form such as tablets, capsules and powders. Powders (such as powders for oral solution) are typically packaged in a sachet or a stick-pack. Alternatively, powders may be filled into two-piece capsules (e.g. gelatine capsules size 0, 00 or 000). In a preferred embodiment of the invention, the term “pharmaceutical dosage form” refers to a solid oral dosage form selected from the group consisting of tablets, capsules and powders. In an even more preferred embodiment of the invention, the term “pharmaceutical dosage form” refers to a tablet or to a capsule. In the most preferred embodiment of the invention, the term “pharmaceutical dosage form” refers to a compressed tablet. Preferably, the tablet of the present invention has a weight of less than 2500 mg, more preferably of less than 2000 mg, and most preferably of less than 1800 mg. Similarly, the capsule of the present invention has preferably a weight of less than 1600 mg, more preferably of less than 1200 mg, and most preferably of less than 1000 mg.

Microcrystalline cellulose (MCC) is a well-known excipient prepared by acid hydrolysis of cellulose. On the industrial scale, MCC is obtained by hydrolysis of wood and/or cotton cellulose using dilute mineral acids. The treated pulp is then rinsed and spray-dried with or without an additional process step such as milling. Numerous types of microcrystalline cellulose (MCC) are available on the market. In the context of the present invention, the term “microcrystalline cellulose” includes any type of microcrystalline cellulose consisting of partially depolymerized cellulose such as the excipients listed in Table 1 of T. Vehovec et al.: “Influence of different types of commercially available microcrystalline cellulose on degradation of perindopril erbumine and enalapril maleate in binary mixtures”, Acta Pharm. 62 (2012), page 518. Excluded is silicified microcrystalline cellulose such as PROSOLV® SMCC. In the context of the present invention, the term “silicified microcrystalline cellulose” refers to an excipient comprising microcrystalline cellulose (MCC) and silicon dioxide such as colloidal silicon dioxide (CSD).

### Pharmaceutical Dosage Form

The present invention relates to a pharmaceutical dosage form comprising:
a) metformin or a pharmaceutical acceptable salt thereof,
b) calcium citrate, and
c) vitamin B12.

A preferred embodiment of the invention relates to a solid oral dosage form comprising:
a) metformin or a pharmaceutical acceptable salt thereof,
b) calcium citrate, and
c) vitamin B12.

A more preferred embodiment of the invention relates to a tablet or a capsule comprising:
a) metformin or a pharmaceutical acceptable salt thereof,
b) calcium citrate, and
c) vitamin B12
   wherein said tablet or said capsule comprises preferably from 0.0001 to 0.012 mol Ca²⁺, more preferably from 0.0003 to 0.006 mol Ca²⁺ and most preferably from 0.0005 to 0.001 mol Ca²⁺.

In the present invention, metformin HCl is the preferred pharmaceutical acceptable salt of metformin. Thus, a preferred embodiment of the invention relates to a tablet, capsule or powder comprising:
a) 500 mg metformin HCl or 1000 mg metformin HCl,
b) anhydrous calcium citrate, tricalcium dicitrate tetrahydrate or a mixture thereof, and
c) optionally vitamin B12.

In the present invention, calcium citrate refers preferably to anhydrous calcium citrate, tricalcium dicitrate tetrahydrate or a mixture thereof. Thus, a preferred embodiment of the invention, relates to a tablet or to a capsule comprising:
a) metformin HCl,
b) anhydrous calcium citrate, tricalcium dicitrate tetrahydrate or a mixture thereof, and
c) vitamin B12
   wherein said tablet or said capsule comprises preferably from 0.0001 to 0.012 mol Ca²⁺, more preferably from 0.0003 to 0.006 mol Ca²⁺ and most preferably from 0.0005 to 0.001 mol Ca²⁺.

During compression of a tablet, capping can be avoided or at least reduced when using granulated metformin. Therefore, the present invention also relates to a tablet comprising:
a) granulated metformin, preferably granulated metformin HCl,
b) anhydrous calcium citrate, tricalcium dicitrate tetrahydrate or a mixture thereof, and
c) vitamin B12.

In the present invention, vitamin B12 refers preferably to cyanocobalamin. Thus, a preferred embodiment of the invention relates to a tablet or to a capsule comprising:
a) 500 mg metformin HCl or 1000 mg metformin HCl,
b) anhydrous calcium citrate, tricalcium dicitrate tetrahydrate or a mixture thereof, and
c) cyanocobalamin
   wherein said tablet or said capsule comprises preferably from 0.0001 to 0.012 mol Ca²⁺, more preferably from 0.0003 to 0.006 mol Ca²⁺ and most preferably from 0.0005 to 0.001 mol Ca²⁺.

Content uniformity of vitamin B12 can be drastically improved by using a spray dried formulation of vitamin B12. Thus, a preferred embodiment of the invention relates to a tablet, capsule or powder comprising:
a) metformin HCl,
b) anhydrous calcium citrate, tricalcium dicitrate tetrahydrate or a mixture thereof, and
c) at least one spray dried formulation of vitamin B12, preferably at least one spray dried formulation of cyanocobalamin.

An also preferred embodiment of the invention relates to a tablet comprising:
a) granulated metformin HCl,
b) anhydrous calcium citrate, tricalcium dicitrate tetrahydrate or a mixture thereof, and
c) at least one spray dried formulation of cyanocobalamin
   wherein said tablet comprises preferably from 0.0001 to 0.012 mol Ca²⁺, more preferably from 0.0003 to 0.006 mol Ca²⁺ and most preferably from 0.0005 to 0.001 mol Ca²⁺.

An also preferred embodiment of the invention relates to a tablet comprising at least one granulate, wherein said granulate comprises:
a) metformin HCl,
b) anhydrous calcium citrate, tricalcium dicitrate tetrahydrate or a mixture thereof, and
c) at least one spray dried formulation of cyanocobalamin.

Surprisingly, tablet hardness can be improved when using tricalcium dicitrate tetrahydrate instead of anhydrous calcium citrate. Therefore, a preferred embodiment of the invention relates to a tablet comprising:
a) metformin or a pharmaceutical acceptable salt thereof,
b) tricalcium dicitrate tetrahydrate, and
c) vitamin B12
   wherein said tablet has preferably a weight of less than 2500 mg, more preferably of less than 2000 mg, and most preferably of less than 1800 mg.

A more preferred embodiment of the invention relates to a tablet comprising:
a) metformin HCl,
b) tricalcium dicitrate tetrahydrate, and
c) vitamin B12, preferably cyanocobalamin.

An also preferred embodiment of the invention relates to a tablet comprising:
a) metformin HCl, preferably granulated metformin HCl,
b) tricalcium dicitrate tetrahydrate, and
c) at least one spray dried formulation of cyanocobalamin.

An even more preferred embodiment of the invention relates to a tablet comprising:
a) metformin HCl, preferably granulated metformin HCl,
b) tricalcium dicitrate tetrahydrate, and
c) at least one spray dried formulation of vitamin B12,
wherein said spray dried formulation of vitamin B12 is preferably a powder which is obtainable by spray drying an aqueous solution which comprises cyanocobalamin and at least one excipient, and
wherein said at least one excipient is preferably selected from the group consisting of sodium citrate, trisodium citrate, citric acid, maltodextrin and modified food starch, and
wherein said powder comprises preferably 1 weight-% or less of cyanocobalamin, based on the total weight of the powder.

The present invention also relates to the use of tricalcium dicitrate tetrahydrate for increasing the hardness of a tablet which comprises metformin or a pharmaceutical acceptable salt thereof, wherein tricalcium dicitrate tetrahydrate is added to said tablet.

Surprisingly, tablet hardness can be further improved when using microcrystalline cellulose instead of silicified microcrystalline cellulose.

Therefore, a preferred embodiment of the invention relates to a tablet comprising:
a) metformin or a pharmaceutical acceptable salt thereof,
b) tricalcium dicitrate tetrahydrate,
c) optionally vitamin B12, and
d) microcrystalline cellulose,
   wherein said tablet comprises less than 1 weight-%, preferably less than 0.5 weight-% and most preferably less than 0.1 weight-% silicified microcrystalline cellulose, based on the total weight of the tablet, and wherein said tablet is preferably free of silicified microcrystalline cellulose.

Thus, the most preferred embodiment of the invention relates to a tablet comprising:
a) metformin HCl, preferably granulated metformin HCl,
b) tricalcium dicitrate tetrahydrate,
c) at least one spray dried formulation of vitamin B12, and
d) microcrystalline cellulose, and
wherein said tablet comprises less than 1 weight-%, preferably less than 0.5 weight-% and most preferably less than 0.1 weight-% silicified microcrystalline cellulose, based on the total weight of the tablet, and
wherein said spray dried formulation of vitamin B12 is a powder which is obtainable by spray drying an aqueous solution which comprises cyanocobalamin and at least one excipient, and
wherein said is excipient is preferably selected from the group consisting of sodium citrate, trisodium citrate, citric acid, maltodextrin and modified food starch, and
wherein said powder comprises preferably 1 weight-% or less of cyanocobalamin, based on the total weight of the powder.

A less preferred embodiment of the invention relates to a liquid. A preferred liquid oral dosage form comprises:
a) metformin or a pharmaceutical acceptable salt thereof,
b) anhydrous calcium citrate, tricalcium dicitrate tetrahydrate or a mixture thereof, and
c) vitamin B12.

Such liquid oral dosage form has good storage stability due to the properties of the chosen calcium citrate.

Method of Preparing the Pharmaceutical Dosage Form

In one embodiment of the invention, the pharmaceutical dosage form is a powder. Such powder can be prepared by mixing the components of the powder. Mixing is necessary for achieving the required content uniformity. Independent of the chosen mixing method, content uniformity is improved if a spray dried formulations of vitamin B12 is used. Suitable spray dried formulation of vitamin B12 are commercially available as “Vitamin B12 1% SD” or “Vitamin B12 0.1% WS” from DSM® Nutritional Products. In the context of the present invention, commercially available “Vitamin B12 0.1% WS” is the preferred spray dried formulation of vitamin B12. Therefore, a preferred embodiment of the present relates a method of preparing a pharmaceutical dosage form, said method comprising the step:
a) providing a powder by mixing metformin or a pharmaceutical acceptable salt thereof, calcium citrate, at least one spray dried formulation of vitamin B12 and preferably at least one excipient.

In another embodiment of the invention, the pharmaceutical dosage form is liquid or is a powder for preparing a liquid oral solution. For preparing a liquid pharmaceutical dosage form, crystalline vitamin B12 can be used. Thus, the present invention also relates to a liquid comprising metformin, vitamin B12, calcium citrate and water, wherein said liquid obtainable by dissolving and/or dispersing crystalline vitamin B12, at least one source of metformin and at least one source of calcium citrate in water.

Preferably, the pharmaceutical dosage form of the invention is a tablet. Such tablet can be prepared by a method comprising the steps:
a) providing a mixture comprising metformin or a pharmaceutical acceptable salt thereof, calcium citrate and preferably at least one excipient,
b) compressing the mixture of step a) into a tablet.

A preferred method comprises the steps:
a) providing a mixture comprising metformin or a pharmaceutical acceptable salt thereof, calcium citrate, vitamin B12 and preferably at least one excipient,
b) compressing the mixture of step a) into a tablet.

A more preferred method comprises the steps:
a) providing a mixture comprising metformin or a pharmaceutical acceptable salt thereof, tricalcium dicitrate tetrahydrate, at least one spray dried formulation of vitamin B12 and preferably at least one excipient,
b) compressing the mixture of step a) into a tablet.

Tablets prepared by this method have suitable hardness. To further increase hardness of the tablet, microcrystalline cellulose may be added. Thus, an also preferred method comprises the steps:
a) providing a mixture comprising metformin or a pharmaceutical acceptable salt thereof, calcium citrate, vitamin B12 and microcrystalline cellulose,
b) compressing the mixture of step a) into a tablet.

When compressing tablets, capping is sometimes observed. The term capping is used when either the upper or lower part of the tablet separates horizontally either partially away from the main body or completely to form a cap when ejected from the press or during the handling process. Capping can be avoided by granulation prior to compression. Thus, a preferred method of preparing the pharmaceutical dosage form of the invention comprises the steps:
a) providing a mixture comprising metformin or a pharmaceutical acceptable salt thereof, calcium citrate, vitamin B12 and at least one pharmaceutical acceptable excipient,
b) granulation of the mixture of step a)
c) compressing the mixture of step b) into a tablet.

Thus, capping can be avoided by inserting an additional process step before compressing the tablet. Alternatively, and preferred, capping can be avoided by using granulated metformin or a granulated pharmaceutical acceptable salt of metformin. Thus, a preferred method of preparing the pharmaceutical dosage form of the invention comprises the steps:
a) providing a mixture comprising granulated metformin HCl, calcium citrate, vitamin B12 and at least one excipient,
b) compressing the mixture of step a) into a tablet.
   wherein said excipient is preferably microcrystalline cellulose.

Use of Calcium Citrate

so Metformin induced vitamin B12 deficiency and metformin induced vitamin B12 malabsorption may be prevented, treated or alleviated by providing calcium ions to the intestinal site where absorption of vitamin B12 is supposed to take place (i.e. in the ileum). Unfortunately, in case of oral administration of calcium carbonate, a significant part of the carbonate's calcium ions will not be available in the ileum due to unwanted precipitation.

The inventors of the present invention have found that in SIF (simulated intestinal fluid, pH=6.8, prepared according to Ph.Eur.), calcium citrate has a better solubility than other calcium salts. According to Fallingborg, the pH gradually increases in the small intestine from pH 6 to about pH 7.4 in the terminal ileum (“Intraluminal pH of the human gastrointestinal tract”, Dan Med Bull. 1999 June; 46(3):183-96). Therefore, precipitation of calcium ions in the human ileum is less likely to happen if calcium citrate is administered.

Accordingly, one embodiment of the invention relates to the use of calcium citrate for preventing the precipitation of calcium ions in the human ileum after intake of a pharmaceutical composition comprising at least one calcium salt and metformin. A preferred embodiment of the invention relates to the use of anhydrous calcium citrate, tricalcium dicitrate tetrahydrate, or a mixture thereof for preventing the precipitation of calcium ions in the human ileum after intake of a pharmaceutical composition comprising at least one calcium salt and metformin. An even more preferred embodiment of the invention relates to the use of anhydrous calcium citrate, tricalcium dicitrate tetrahydrate, or a mixture thereof for preventing the precipitation of calcium ions in the human ileum after intake of a pharmaceutical composition comprising at least one calcium salt, metformin and vitamin B12.

An alternative embodiment of the invention relates to the use of calcium citrate in a pharmaceutical dosage form for preventing the precipitation of calcium ions in simulated intestinal fluid and/or in the human ileum. An also preferred embodiment of the invention relates to the use of calcium citrate in a pharmaceutical dosage form for preventing the precipitation of calcium ions which are moving within a mammal, preferably within a human being, from the jejunum into the ileum.

Method of Treatment

The present invention also relates to a method for the treatment or prevention of metformin induced vitamin B12 deficiency, said method comprising the step of administering the herein described pharmaceutical dosage form. Thus, a preferred embodiment of the invention relates to a method for the treatment or prevention of metformin induced vitamin B12 deficiency, said method comprising the step of administering a pharmaceutical dosage form comprising:
a) metformin or a pharmaceutical acceptable salt thereof,
b) tricalcium dicitrate tetrahydrate, and
c) vitamin B12, preferably cyanocobalamin.

An alternative embodiment of the invention relates to a composition as herein described for use in the treatment or prevention of metformin induced vitamin B12 deficiency. It also relates to a tablet or to a capsule as herein described for use in the treatment or prevention of metformin induced vitamin B12 deficiency, wherein said tablet or said capsule comprises from 0.0001 to 0.012 mol Ca²⁺, preferably from 0.0003 to 0.006 mol Ca²⁺ and most from 0.0005 to 0.001 mol Ca²⁺. In these alternative embodiments, calcium citrate is preferably anhydrous calcium citrate, tricalcium dicitrate tetrahydrate, or a mixture thereof, and wherein tricalcium dicitrate tetrahydrate is particularly preferred.

The present invention also relates to a method for the prevention or alleviation of metformin induced vitamin B12 malabsorption, said method comprising the step of administering the herein described pharmaceutical dosage form. Thus, a preferred embodiment of the invention relates to a method for the prevention or alleviation of metformin induced vitamin B12 malabsorption, said method comprising the step of administering a pharmaceutical dosage form comprising:
a) metformin or a pharmaceutical acceptable salt thereof,
b) tricalcium dicitrate tetrahydrate, and
c) optionally vitamin B12, preferably cyanocobalamin.

An alternative embodiment relates to a pharmaceutical dosage form as herein described for use in the prevention or alleviation of metformin induced vitamin B12 malabsorption. It also relates to a tablet or to a capsule as herein described for use in the prevention or alleviation of metformin induced vitamin B12 malabsorption, wherein said tablet or said capsule comprises from 0.0001 to 0.012 mol Ca²⁺, preferably from 0.0003 to 0.006 mol Ca²⁺ and most preferably from 0.0005 to 0.001 mol Ca²⁺. In these alternative embodiments, calcium citrate is preferably anhydrous calcium citrate, tricalcium dicitrate tetrahydrate, or a mixture thereof, and wherein tricalcium dicitrate tetrahydrate is particularly preferred.

Long-term treatment with metformin has been associated with a decrease in serum vitamin B12 levels which may cause peripheral neuropathy. Therefore, the present invention also relates to a method for the prevention of metformin induced peripheral neuropathy, said method comprising the step of administering the herein described pharmaceutical dosage form. Thus, a preferred embodiment of the invention relates to a method for the prevention of metformin induced peripheral neuropathy, said method comprising the step of administering a pharmaceutical dosage form comprising:
a) metformin or a pharmaceutical acceptable salt thereof,
b) tricalcium dicitrate tetrahydrate, and
c) optionally vitamin B12, preferably cyanocobalamin.

An alternative embodiment relates to a pharmaceutical dosage form as herein described for use in the prevention of metformin induced peripheral neuropathy. It also relates to a tablet or to a capsule as herein described for use in the prevention of metformin induced peripheral neuropathy wherein said tablet or said capsule comprises from 0.0001 to 0.012 mol Ca²⁺, preferably from 0.0003 to 0.006 mol Ca²⁺ and most preferably from 0.0005 to 0.001 mol Ca²⁺. In these alternative embodiments, calcium citrate is preferably anhydrous calcium citrate, tricalcium dicitrate tetrahydrate, or a mixture thereof, and wherein tricalcium dicitrate tetrahydrate is particularly preferred.

### EXAMPLES

### Example 1

First Trial

Three similar tablet mixtures were prepared, each comprising metformin HCl, a spray dried formulation of vitamin B12 (available at DSM® Nutritional Products), a binder and a calcium salt. Thereby, three different calcium salts were used: calcium carbonate, calcium phosphate and tricalcium dicitrate tetrahydrate, respectively. In all three cases, capping was observed. Thus, none of the three tablet mixtures of the first trial could be successfully compressed into a tablet.

Second Trial

In a second trial, three tablet mixtures were prepared, similar to the first trial. This time, however, a granulated metformin (Metformin DC 92.6%, granulated with magnesium stearate as lubricant, available at Vistin Pharma) was used instead of non-granulated metformin HCl. In this second trial, no capping was observed, regardless which calcium salt had been used.

Example 1 illustrates that granulated metformin is preferably used in case a tablet is to be compressed. Alternatively, non-granulated metformin is mixed with calcium citrate and the other optional compounds. The thus obtained mixture is then granulated before the actual tabletting process.

### Example 2

In example 2, four similar tablet mixtures were prepared, each comprising granulated metformin DC 92.6%, a spray dried formulation of vitamin B12 (available at DSM® Nutritional Products), Aerosil 200 as a flowing agent, magnesium stearate as a lubricant, ProsoIv® SMCC90 (available at JRS Pharma) as a binder, and a calcium salt. Thereby, four different kinds of calcium salts were tested: calcium carbonate (95MD available at Particle Dynamics), dicalciumphosphat anhydrous (DiCafos A150, anhydrous, available at Budenheim), tricalcium dicitrate tetrahydrate (available at Merck) and anhydrous calcium citrate (available at Gadot).

For compressing the tablets, a single punch press (Korsch XP-1, available at Korsch, Berlin) was used.

All four tablet mixtures could be successfully compressed into a tablet, regardless which calcium salt had been used. Each of the tablet comprised the same amount of metformin, vitamin B12 (spray dried formulation), Ca²⁺ (100 mg/tablet), flowing agent and lubricant. The amount of binder (ProsoIv® SMCC90) per tablet was then chosen such that each of the obtained tablet had a mass of 1500 mg.

Tablet hardness was then measured using a Krämer UTS4 1 apparatus. The obtained compression profiles are shown in FIG. 1.

FIG. 1 shows that the hardness of the tablet depends on the chosen calcium salt. Hardest tablets are achieved when using tricalcium dicitrate tetrahydrate. Surprisingly, tablet hardness is a lot worse when using anhydrous calcium citrate.

Good hardness was also achieved for calcium carbonate. However, calcium carbonate has the disadvantage of low solubility in simulated intestinal fluid and/or bioavailability. In addition, perception by patients of calcium carbonate is negative. Dicalciumphosphat anhydrous did not perform as good as tricalcium dicitrate tetrahydrate.

### Example 3

In order to further improve tablet hardness, four similar tablet mixtures were prepared as shown in below TABLE 1:

TABLE 1

| | tablet | tablet | tablet | tablet |
|---|---|---|---|---|
| | mixture 3a | mixture 3a′ | mixture 3b | mixture 3b′ |
| Ca-source | tricalcium | tricalcium | Calcium | Calcium |
| | dicitrate | dicitrate | carbonate² | carbonate² |
| | tetrahydrate¹ | tetrahydrate¹ | | |
| binder | Avicel | Prosolv ® | Avicel | Prosolv ® |
| | PH102³ | SMCC90⁴ | PH102³ | SMCC90⁴ |
| metformin | Metformin | Metformin | Metformin | Metformin |
| | DC 92.6% | DC 92.6% | DC 92.6% | DC 92.6% |
| vitamin B12 | spray dried | spray dried | spray dried | spray dried |
| | formulation | formulation | formulation | formulation |
| | from DSM ® | from DSM ® | from DSM ® | from DSM ® |
| flowing agent | Aerosil 200 | Aerosil 200 | Aerosil 200 | Aerosil 200 |
| lubricant | magnesium | magnesium | magnesium | magnesium |
| | stearate | stearate | stearate | stearate |

¹tricalcium dicitrate tetrahydrate (available at Merck)
²calcium carbonate (95MD available at Particle Dynamics)
³Avicel ® PH102 (available at FMC Biopolymer)
⁴Prosolv ® SMCC90 (available at JRS Pharma)

Said four tablet mixtures were compressed into tablets, similar to example 2. Tablet hardness was then measured. The result is shown in FIGS. 2a and 2b.

FIG. 2a shows the compression curves of tablets, comprising tricalcium dicitrate tetrahydrate as source of Ca²⁺ and as binder Avicel® PH102 (3a) or ProsoIv® SMCC90 (3a′).

FIG. 2b shows the compression curves of tablets, comprising calcium carbonate as source of Ca²⁺ and as binder Avicel® PH102 (3b) or ProsoIv® SMCC90 (3b′).

Example 3 shows that the hardness of tablets comprising metformin can be further improved by using microcrystalline cellulose as binder, provided the tablet comprises calcium citrate as source of ionic calcium. Surprisingly, if calcium carbonate is used instead of calcium citrate, tablet hardness cannot be improved by replacing silicified microcrystalline cellulose with microcrystalline cellulose.

### Example 4

In Example 4, the solubility of four different calcium salts was analysed in three different dissolution media via determination of the Ca²⁺ ions content by ICP-OES (inductively coupled plasma optical emission spectrometry).

The calcium salts were:
calcium carbonate (95MD available at Particle Dynamics),
dicalciumphosphat anhydrous (DiCafos A150, available at Budenheim),
tricalcium dicitrate tetrahydrate (available at Merck)
anhydrous calcium citrate (available at Gadot).

The solubilization media were:
water,
SGF (simulated gastric fluid, pH=1.1, prepared according to Ph. Eur.)
SIF (simulated intestinal fluid, pH=6.8, prepared according to Ph. Eur.)

All three dissolution media were heated to 37° C. and the analyses were performed at this temperature. During the analyses, the salts were added in access to the solubilization media and left to mix for 24 h. Afterwards, the solutions were filtered, and the precipitates were investigated using ATR-IR analysis to confirm the presence of the starting material (e.g. respective Ca salt). The filtered solutions were analysed for Ca²⁺ ions content. The solubility results that were obtained are shown in the TABLES 2, 3 and 4.

In all three media, calcium carbonate shows the poorest solubility. Thus, the amount of calcium ions taught by Bauman et al. can be reduced if a calcium salt other than calcium carbonate is administered.

In SGF (simulated gastric fluid), solubility of dicalcium phosphate is somewhat similar to the solubility of the two calcium citrate salts. Thus, it can be assumed that in all three cases, calcium ions will be available in stomach of the patient. However, it is not the patient's stomach that matters.

For absorption of vitamin B12, a vitamin B12-IF complex is formed which then binds to enterocyte receptors in the ileum. For this process in the patient's ileum, the presence of ionic calcium is obligatory. Thus, it is in the ileum where calcium ions are needed.

In SIF (simulated intestinal fluid), solubility of the two calcium citrate salts is significantly higher than solubility of dicalcium phosphate and calcium carbonate. Thus, vitamin B12 malabsorption can be effectively reversed if calcium citrate (instead of calcium carbonate or dicalcium phosphate) is orally administered. “Effectively” can mean that an amount of calcium salt smaller than suggested by Bauman et al. is sufficient to reverse metformin induced vitamin B12 malabsorption.

TABLE 2

| | water | | | | |
|---|---|---|---|---|---|
| | | solubility, | | | Ca²⁺ ions, |
| | solubility | average, | | Ca²⁺ ions | average, |
| | mg/ml | mg/ml | pH | mg/ml | mg/ml |
| Ca carbonate | 0.019 | 0.019 | 9.5 | 0.0075 | 0.0075 |
| | 0.019 | | 9.5 | 0.0075 | |
| dicalcium | 0.177 | 0.180 | 6.0 | 0.0520 | 0.0530 |
| phosphate | 0.182 | | 6.0 | 0.0540 | |
| anhydrous | | | | | |
| tricalcium | 0.916 | 0.914 | 5.9 | 0.1900 | 0.1900 |
| dicitrate | 0.911 | | 5.9 | 0.1900 | |
| tetrahydrate | | | | | |
| anhydrous | 0.846 | 0.843 | 6.6 | 0.2000 | 0.2000 |
| calcium | 0.839 | | 6.5 | 0.2000 | |
| citrate | | | | | |

TABLE 3

| | SGF | | | | |
|---|---|---|---|---|---|
| | | solubility | | | Ca²⁺ ions, |
| | solubility | mg/ml | | Ca²⁺ ions | mg/ml, |
| | mg/ml | average | pH | mg/ml | average |
| Ca carbonate | 4.5 | 4.59 | 7.2 | 1.8 | 1.85 |
| | 4.7 | | 7.1 | 1.9 | |
| dicalcium | 10.4 | 10.35 | 3.0 | 3.1 | 3.05 |
| phosphate | 10.3 | | 3.0 | 3.0 | |
| anhydrous | | | | | |
| tricalcium | 12.5 | 12.50 | 3.3 | 2.6 | 2.6 |
| dicitrate | 12.5 | | 3.3 | 2.6 | |
| tetrahydrate | | | | | |
| anhydrous | 11.0 | 11.05 | 3.3 | 2.7 | 2.7 |
| calcium | 11.1 | | 3.3 | 2.7 | |
| citrate | | | | | |

TABLE 4

| | SIF | | | | |
|---|---|---|---|---|---|
| | | solubility | | | Ca²⁺ ions |
| | solubility | mg/ml | | Ca²⁺ ions | mg/ml, |
| | mg/ml | average | PH | mg/ml | average |
| Ca carbonate | 0.058 | 0.06 | 7.5 | 0.023 | 0.024 |
| | 0.060 | | 7.5 | 0.024 | |
| dicalcium | 0.093 | 0.09 | 6.7 | 0.027 | 0.028 |
| phosphate | 0.094 | | 6.6 | 0.028 | |
| anhydrous | | | | | |
| tricalcium | 2.130 | 2.11 | 6.1 | 0.450 | 0.445 |
| dicitrate | 2.090 | | 6.1 | 0.440 | |
| tetrahydrate | | | | | |
| anhydrous | 1.920 | 1.91 | 6.1 | 0.460 | 0.460 |
| calcium | 1.890 | | 6.1 | 0.460 | |
| citrate | | | | | |

### Example 5

Depending on the medical indication, either an extended release (XR) dosage form or an immediate release (IR) dosage form is prescribed. An example of a commercially available immediate release formulation is Glucophage® IR.

To achieve immediate release, disintegration time of a tablet should be short. In many cases, increased tablet hardness results in longer disintegration time than normal. Surprisingly, this is not the case when tricalcium dicitrate tetrahydrate is used as source of Ca²⁺.

In example 5, the physical characteristics of the four tablets of example 2 were measured. The results are shown in below TABLE 5:

TABLE 5

| | Tablet 1 | Tablet 2 | Tablet 3 | Tablet 5 |
|---|---|---|---|---|
| Calcium salt | dicalciumphosphate | calcium | tricalcium dicitrate | anhydrous |
| | anhydrous | carbonate | tetrahydrate | calcium citrate |
| Tablet thickness (mm) | 7.61 | 7.60 | 7.90 | 8.34 |
| Tablet weight (mg) | 1507 mg | 1502 mg | 1499 mg | 1508 mg |
| Hardness (N) | 252 N | 274 N | 275 N | 194 N |
| Friability (%) | 0.07 | 0.08 | 0.07 | 0.23 |
| Disintegration time (min:s) | 06:06 | 07:00 | 03:07 | 01:07 |

Tablet hardness was measured as described in USP <1217> and EP<2.9.8.> with a Krämer UTS4 1 tester (Kraemer Elektronik GmbH, Darmstadt, Germany). The inventors measured the force needed to break a tablet axially. Presented are the average values of 10 measurements.

Tablet disintegration was characterized according USP<701, 2040> by using a DISI-1 disintegration tester (Charles Ischi PG Pharma Prüftechnik, Zuchwill, Switzerland) in 900 mL demineralized water at 37° C. Six parallel measurements were carried out. Upper limit of disintegration time is 30 min for uncoated tablets (USP <2040>).

Friability, that is closely related to tablet hardness, refers to the extent of weight loss during mechanical abrasion. A maximum loss of no more than 1% of the initial tablet weight is considered acceptable (USP <1216>, EP<2.9.7.>). The inventors tested 10 tablets in an AE-1 Friabilator (Charles Ischi AG Pharma Prüftechnik, Zuchwill, Switzerland) at a rotation speed of 25 rpm for 4 minutes. The weight loss of the tablets was recorded.

Surprisingly, using tricalcium dicitrate tetrahydrate as source of Ca²⁺ results in tablets with (i) high hardness, (ii) short disintegration time and (iii) low friability.

### Example 6

In example 6, three different kinds of tablets were prepared. Each tablet comprised 549.9 mg calcium phosphate (anhydrous, available at Emcompress®) and 0.0078 mg vitamin B12. Apart from the source of vitamin B12, the different kinds of tablets were identical.

To investigate the impact on content uniformity, the following three different kinds of vitamin B12 were tested:
Vitamin B12 cryst. (crystalline vitamin B12, available at DSM® Nutritional Products)
Vitamin B12 1% SD (spray dried formulation of vitamin B12, available at DSM® Nutritional Products)
Vitamin B12 0.1% WS (spray dried formulation of vitamin B12, available at DSM® Nutritional Products)

Tablets were compressed with a Korsch XL 100 rotary tableting machine (Korsch AG, Berlin, Germany) using an oblong punch of 22×9 mm and compression force of 20 kN.

Vitamin B12 content uniformity was then evaluated via the standard deviation RSD (%) calculated from 10 individual assay determinations (HPLC analysis conducted by Eurofins®, Germany).

As shown at below TABLE 6, the relative standard deviations (RSD) values relating to the two spray dried formulations of vitamin B12 were below 5%, indicating acceptable content uniformity and hence homogeneous distribution of Vitamin B12 in the tablets. In contrast, content uniformity relating to vitamin B12 crystalline was extremely poor.

TABLE 6

| | | tablet 1 | tablet 2 | tablet 3 |
|---|---|---|---|---|
| weight of tablet (mg) | | 1560.8 | 1553.9 | 1538.3 |
| calcium phosphate anhydrous | | 549.9 | 549.9 | 549.9 |
| (mg/tablet) | | | | |
| source of vitamin B12 | | vitamin B12 | vitamin B12 | vitamin B12 |
| | | cryst. | 1% SD | 0.1% WS |
| amount of source of vitamin | | 0.0078 | 0.78 | 7.8 |
| B12 (mg/tablet) | | | | |
| amount of vitamin B12 per | | 0.0078 | 0.0078 | 0.0078 |
| tablet (mg/tablet) | | | | |
| vitamin B12 content | RSD | 90.8 | 3.3 | 2.0 |
| uniformity | (%) | | | |
| | rating | extremely | good | good |
| | | poor | | |

### Example 7 (Recipe of Preferred Tablet)

Below TABLE 7 shows the composition of tablets of the invention.

TABLE 7

| | |
|---|---|
| Tablet 1 | Tablet 2 |

| | | | | |
|---|---|---|---|---|
| Metformin DC 92.6% | 863.9 | mg | 863.9 | mg |
| Vitamin B12 (spray-dried) | 1.56 | mg | 1.56 | mg |

| | | | |
|---|---|---|---|
| tricalcium dicitrate tetrahydrate | 434.78 | mg | — |

| | | | |
|---|---|---|---|
| anhydrous calcium citrate | — | 423.73 | mg |

| | | | | |
|---|---|---|---|---|
| Aerosil 200 | 4.28 | mg | 4.28 | mg |
| Magnesium stearate | 2.5 | mg | 2.5 | mg |
| Prosolv SMCC90 | 192.95 | mg | 204 | mg |
| Total weight of tablet | 1500 | mg | 1500 | mg |

The tablets of Table 7 have at least some of the following features:
although the tablets are very hard, disintegration time is short; no capping was observed during tabletting process.
tablet thickness is not higher than usual despite of comprising a significant amount of calcium citrate
the tablets do not negatively impact patient compliance. In comparison to the intake of two or even three separate tablets, the tablets are expected to enhance patient compliance.
oral intake of the tablets is expected to result in an amount of Ca²⁺ ions in the ileum which prevents and/or at least partially reverses metformin induced vitamin B12 malabsorption
oral intake of the tablets results in an amount of Ca²⁺ ions in the ileum which prevents and/or at least partially reverses metformin induced vitamin B12 deficiency
the tablets have high content uniformity meeting pharmaceutical standards

1. Pharmaceutical dosage form comprising:
a) metformin or a pharmaceutical acceptable salt thereof,
b) calcium citrate, and
c) at least one source of vitamin B12.

2. Pharmaceutical dosage form according to claim 1, said pharmaceutical dosage form comprising:
a) granulated metformin, preferably granulated metformin hydrochloride, and/or
b) anhydrous calcium citrate, tricalcium dicitrate tetrahydrate, or a mixture thereof, and/or
c) crystalline vitamin B12 or a spray dried formulation of vitamin B12.

3. Pharmaceutical dosage form according to claim 1, wherein said calcium citrate is tricalcium dicitrate tetrahydrate.

4. Pharmaceutical dosage form according to claim 1, wherein said pharmaceutical dosage form comprises from 0.0001 to 0.012 mol Ca²⁺, preferably from 0.0003 to 0.006 mol Ca²⁺ and most preferably from 0.0005 to 0.001 mol Ca²⁺.

5. Pharmaceutical dosage form according to claim 1, wherein said pharmaceutical dosage form is an oral dosage form, and wherein said oral dosage form is preferably a solid oral dosage form, and wherein said solid oral dosage form is preferably a tablet or a capsule, and wherein said tablet is preferably a compressed tablet.

6. Pharmaceutical dosage form according to claim 1, wherein said pharmaceutical dosage form is a tablet or a capsule, and wherein said tablet or said capsule has preferably a weight of less than 2500 mg, more preferably a weight of less than 2000 mg, and most preferably a weight of less than 1800 mg.

7. Method of preparing a pharmaceutical dosage form, said method comprising the steps:
a) providing a mixture comprising metformin or a pharmaceutical acceptable salt thereof, calcium citrate, at least one source of vitamin B12 and preferably at least one pharmaceutical acceptable excipient,
b) compressing the mixture of step a) into a tablet.

8. Method according to claim 7, wherein said mixture comprises granulated metformin or a granulated pharmaceutical acceptable salt of metformin, and wherein said mixture comprises preferably granulated metformin HCl.

9. Method according to claim 7, wherein said at least one source of vitamin B12 is crystalline vitamin B12 or a spray dried formulation of vitamin B12, and wherein said at least one source of vitamin B12 is preferably a spray dried formulation of vitamin B12.

10. Method according to claim 7, wherein said mixture comprises tricalcium dicitrate tetrahydrate and/or wherein said mixture comprises at least one flowing agent, at least one lubricant and at least one binder, wherein said at least one binder is preferably microcrystalline cellulose.

11. Use of tricalcium dicitrate tetrahydrate for increasing the hardness of a tablet which comprises metformin or a pharmaceutical acceptable salt thereof, wherein tricalcium dicitrate tetrahydrate is added to said tablet.

12. Composition comprising calcium citrate for use in the treatment or prevention of metformin induced vitamin B12 deficiency.

13. Composition comprising calcium citrate for use in the prevention or alleviation of metformin induced vitamin B12 malabsorption or of metformin induced peripheral neuropathy.

14. Composition according to claim 12, wherein said composition further comprises vitamin B12.

15. Composition according to claim 12, wherein said calcium citrate is anhydrous calcium citrate, tricalcium dicitrate tetrahydrate, or a mixture thereof, and wherein said calcium citrate is preferably tricalcium dicitrate tetrahydrate.
